(19) United States
(12) Patent Application Publication
(10) Pub. No.: US 2006/0292562 A1
(43) Pub. Date: Dec. 28, 2006

(54) Methods of identifying genomic and proteomic biomarkers for cystic fibrosis, arrays comprising the biomarkers and methods of using the arrays

(76) Inventors: Harvey B. Pollard, Potomac, MD (US); David Jacobwitz, Bethesda, MD (US); Greg Mueller, Silver Spring, MD (US); Ofer Eidelman, Bethesda, MD (US); Meera Srivastava, Potomac, MD (US); William Guggino, Baltimore, MD (US); Pamela Zeitlin, Baltimore, MD (US)

Correspondence Address: DLA PIPER US LLP;ATTN: PATENT GROUP, 1200 NINETEENTH STREET, NW, WASHINGTON, DC, 20036, US

(21) Appl. No.: 10/515,844

(22) PCT Filed: May 29, 2003
(86) PCT No.: PCT/US03/17012

§ 371 Date: Apr. 24, 2006

Related U.S. Application Data
(60) Provisional application No. 60/383,605, filed on May 29, 2002.

(51) Int. Cl. C12Q 1/68; G01N 33/00

(52) U.S. Cl. 435/6; 436/86; 204/450

(57) ABSTRACT

Cystic fibrosis (CF) is the most common fatal autosomal recessive disease in the U.S. and is principally caused by the DF508 mutation the CFTR gene. The principal site of morbidity and mortality for this disease is the lung. We have used genomic and proteomic methods to identify ubiquitin carboxy terminal hydrolase-1 (UCHL1) as a biomarker for cystic fibrosis. Both gene expression and cognate protein expression are massively upregulated in CF lung epithelial cells. We suggest that this gene can be useful in the assembly of a diagnostic or prognostic chip for CF, or as a target for therapeutic intervention.

This application claims priority to Provisional U.S. Patent Application Ser. No. 60/383,605, filed May 29, 2002, which is incorporated herein by reference in its entirety.

The U.S. Government has a paid-up license in this invention and the right in limited circumstances to require the patent owner to license others on reasonable terms as provided for by the terms of Grant No. NO-1-HHLBI-HL-02-04 awarded by the National Institutes of Health.

### BACKGROUND OF THE INVENTION

1. Field of the Invention

The present invention is directed to bioassays generally and, in particular, to methods of identifying protein and nucleic acid markers for cystic fibrosis (CF), arrays comprising the biomarkers, and the use of these arrays for the diagnosis and prognosis of CF as well as the screening of potential drugs for the treatment of CF.

2. Background of the Technology

Cystic Fibrosis (CF), which is caused by a mutation in the CFTR gene, is the most common autosomal recessive lethal disease in the United States (Welsh et al. 1995; Welsh et al. 2001). Approximately 5% of the population carries one mutant CFTR gene (Rommens et al., 1989; Riordan et al. 1989; Kerem et al. 1989), and the disease occurs in a frequency of 1 in 2500 live births. Statistically, death occurs in the majority of patients by age 28. At the present time the respiratory difficulties and ensuing complications of inflammation and lung infection are directly responsible for the eventual death of over 90% of CF patents.

The principal CFTR gene mutation, [ΔF508]CFTR, causes preferential ubiquitinylation and proteosomal destruction of the mutant protein. The CFTR, the gene product of the CFTR gene, is a chloride channel protein which trafficks from the endoplasmic reticulum, through the golgi, terminating at the apical plasma membrane of epithelial cells in lung, liver, pancreas, GI tract and elsewhere. In response to an increase in cAMP, protein kinase A phosphorylates the protein and activates the channel activity. The wildtype protein has a plethora of other activities and interactions which may be associated with the disease phenotype. However, the [ΔF508]CFTR mutation, responsible for up to 70% of all cystic fibrosis patients and up to 90% of all CF chromosomes, results in mis-trafficking of the mutant protein and destruction by a ubiquitin-dependent proteosomal system. Failure of the mutant CFTR to be delivered to the apical plasma membrane of epithelial cell results in cystic fibrosis (see Welsh et al. 2001 for a complete review).

The ubiquitin system for destruction of mutant CFTR operates as follows (Hershko and Ciechanover 1998; Voges et al. 1999; Plemper and Hammond 2002). Ubiquitin, an ˜8 KDa protein, is activated by the ubiquitin-activating enzyme E1, and is transferred to the ubiquitin-conjugating enzyme E2. The ubiquitin ligase E3 transfers the ubiquitin to the substrate protein target. In this case the target is mutant CFTR. Thereafter, the substrate becomes poly-ubiquitinylated by clusters of tandem ubiquitins. Upon entry into the 26S proteosome, the ubiquitins are hydrolyzed from the targeted substate by DUB's (deubiquitinylating enzymes). The free ubiquitins are now available for reuse by the system for ubiquitinylating additional substrate proteins. The function of DUB's is therefore to stimulate protein degradation by maximizing the availability of free ubiquitins.

Inflammation in the CF lung is due to a defect in the TNFα/NFκβ signaling pathway, resulting in massively elevated levels of proinflammatory cytokine IL-8. Therefore, elevated levels of proinflammatory factors, including interleukin-8 (IL-8), characterize the lung of CF patients (Bonfield et al. 1995a; Bonfield et al. 1995b). High levels of IL-8 even characterize the lungs and meconium of CF newborns having no objective evidence of infection (Khan et al. 1995; Briars et al. 1995). More recently we have determined that cultured CF lung epithelial cell line IB-3 secrete high levels of IL-8. By contrast, repair of these cells with wildtype CFTR, using AAV-mediated gene therapy, result in profound suppression of IL-8 secretion (Eidelman et al. 2001).

Pharmacogenomic analysis has revealed that the genes principally involved in the CF phenotype are from the TNFα/NFkβ signaling pathway. The mechanism by which this pathway promotes expression of IL-8 is by phosphorylation of IκB by the IkappaBkinase kinase (IKKα and β/NEMO complex; D'Acquisto et al. 2002). The Iκβ is normally complexed to NFκβ[p65] and NFκβ[p50] as an inactive species in the cytosol. However, upon phosphorylation of IκB, the IκB becomes ubiquitinylated and proteolyzed in the proteosome. The E3 ubiquitin ligase Skp1/Cul1/F-box protein, FWD1 (SCF^{β-TRCP}) recruits phosphorylated IκB for ubiquitinylation by the E2 ubiquitin conjugating enzyme Cdc34 (Yaron et al. 1998; Hatakeyama et al. 1999; Kitagawa et al. 1999; Winston et al. 1999). The released NFκβ[p65]/NFκβ[p50] complex is now free to migrate into the nucleus, bind to κβ cis acting sites on the IL-8 and other related promoters, and thereby drive the production of IL-8 message and protein. Therefore, CF lung epithelial cells are characterized by high levels of IL-8 message and cognate protein.

The discovery of additional genes and proteins with CF-specific properties would be desirable since these CF specific genes and proteins could be used as biomarkers for the disease. For example, the cDNA of these genes could be used as a probe on an array (e.g., a microarray) for use in the diagnosis or prognosis of CF.

### SUMMARY OF THE INVENTION

According to a first aspect of the invention, a method of determining the level of expression of a population of proteins in a first cell is provided comprising: growing first cells in a medium comprising radio-labeled amino acids such that radio-labeled amino acid is incorporated into the proteins; lysing the first cells; placing the first cell lysate on a first gel; separating the proteins in the first cell lysate using 2-D gel electrophoresis; and imaging the first gel using autoradiography. The first cells can have a mutated form of the CFTR gene. In addition, the method can further include: growing second cells having the wildtype CFTR gene in methionine-free medium supplemented with 35[S] methionine; lysing the second cells; placing the second cell lysate on a second gel; separating the proteins in the second cell lysate using 2-D gel electrophoresis; imaging the second gel using autoradiography; and comparing the images for the first and second cells. The first cells can be IB-3 cells and the second cells can be IB-3/S9 cells. The method can further include identifying proteins that exhibit different levels of expression between the first and second cells. The method can also include identifying the nucleotide sequence of aptamers which bind the identified proteins and constructing an array comprising aptamers having the identified sequences attached to a solid support. The radio-labeled amino acid can be 35[S] methionine. The population of proteins can be the proteome of the first cell.

According to a second aspect of the invention, an array comprising a plurality of different probes disposed on a surface of a solid support is provided wherein each of the different probes bind to a different marker for cystic fibrosis. The plurality of different probes can include probes for UCHL-1 and IL-8. The probes and markers can be nucleic acids. For example the probes can be cDNA or oligonucleotide probes and the markers can be mRNA. Alternatively, the probes can be nucleic acids (e.g., aptamers) and the markers can be proteins. The markers can be selected from the group consisting of: NMDA Receptor subunit epsilon 2 (NMDAR2B); Voltage gated potassium channel protein KV12; Leukocyte common antigen (L-CA; CD45 antigen); Adenosine A1 Receptor (ADORA1); CD40 Receptor Associated Antigen (CRAF-1); Tumor Necrosis Factor alpha; parkin; glutathione S-Transferase A1 (GTH1); Signal transducer and activator of transcription 1 (STAT1); ergB; DNA binding protein HIP116; Bone Morphogenic Protein3 (BMP3); translin; PI3-Kinase, p110; IL-2Rgamma; cmyc oncogene; lissencephalin X; cAMP Response Element Binding Protein (CREBBP); casein kinase 1 gamma 2; ribosomal protein S6 kinase II alpha 3; macrophage-specific colony stimulating factor (MCSF); cellular retinoic acid binding protein II (CRABP2); cadherin 3 (P-cadherin); basic transcription factor 62-kDa subunit (BTF2); placenta growth factor 1; placenta growth factor 2; FUSE binding protein; leukemia inhibitory factor (LIF; HILDA); beta-interferon gene positive regulatory domain 1 binding factor (BLIMP1); interferon consensus sequence-binding protein (ICSBP); calcium activated potassium channel HSK1; NFkB, p100 (NFkB, p52); IL-17; GABA Receptor epsilon subunit [GABA(A)Receptor]; RAB3B; p16-INK4; frizzled; OCT-2; IL-4; Matrix metalloproteinase 12 (MMP12); G-Protein activated inward rectifier Potassium channel 3 (KIR3.3); zinc finger protein 91; DNA Repair protein XRCC1; RAG2; IL-8; actophilin; coactosin; UCH-L1 and combinations thereof.

According to a third aspect of the invention, a method is provided including: removing cells from a patient; lysing the cells; and contacting the cell lysate with an array as set forth above. The method can further include imaging the array. The patient can be a patient that has not been diagnosed with cystic fibrosis wherein the method is a method for diagnosing cystic fibrosis. The method can further include comparing the image of the array with a control image made by imaging an array contacted with a control composition comprising the lysate of a cell having the wildtype CFTR gene. Alternatively, the patient can be a patient that has been diagnosed with cystic fibrosis wherein the method is a method for determining the prognosis of the disease. According to a further embodiment, the patient can be a patient undergoing treatment for cystic fibrosis wherein the method is a method for determining the effectiveness of the treatment. The method can further include comparing the image of the array with a control image made by imaging an array contacted with a control composition comprising the lysate of a cell sample removed from the patient during an earlier stage of the treatment.

According to a fourth aspect of the invention, a method is provided including: contacting cells having a mutated form of the CFTR gene with a composition comprising a test compound; lysing the cells; and contacting the cell lysate with an array as set forth above. This method can further include imaging the array and comparing the image of the array with a control image made by imaging an array contacted with a control composition which does not include the test compound.

### BRIEF DESCRIPTION OF THE DRAWINGS

The present invention may be better understood with reference to the accompanying drawing in which:

FIG. 1A-1D are 2-D gels of lung epithelial CF IB-3 (FIGS. 1A and 1C) and wildtype CFTR-repaired IB-3/S9 (FIGS. 1B and 1D) cell proteomes wherein the gels shown in FIGS. 1A and 1B were imaged with silver stain and wherein the proteomes imaged in the gels shown in FIGS. 1C and 1D were labeled with 35 [S] methionine and imaged in the 3rd dimension as an autoradiogram;

FIGS. 2A and 2B are density maps of identical regions of a gel stained with silver (FIG. 2A) and 35[S] methionine (FIG. 2B);

FIGS. 3A-3C illustrate the isolation and identification of the CF-specific protein, ubiquitin carboxy terminal hydrolase-1 (UCHL1), by 35[S] methionine labeling and identification by mass spectrometry wherein FIGS. 3A and 3B are 2-D gels of wildtype CFTR-repaired IB-3/S9 cells and CF IB-3 cells, respectively, each imaged in the 3^{rd} dimension as an autoradiogram wherein the circle in each figure marks a feature present in the CF cell but not in the repaired IB-3/S9 cell and wherein FIG. 3C is a mass spectrogram of the feature circled in FIG. 3B;

FIGS. 4A and 4B are imaged cDNA arrays showing gene expression of UCHL1 wherein the sample assayed in FIG. 4A is RNA from the cystic fibrosis cell line IB-3 and the sample assayed in FIG. 4B is RNA from the wildtype CFTR-repaired cell line IB-3/S9 (FIG. 4B);

FIG. 5 is a Western blot image by enhanced chemiluminescence (ECL) of UCHL-1 protein in IB-3 cells and IB-3/S9 cells; and

FIG. 6 is a schematic illustrating a method of identifying potential CF markers according to an embodiment of the invention.

### DETAILED DESCRIPTION

By identifying molecular differences between control cells and those expressing a CF mutation, one can delineate biomarkers or therapaeutic targets for CF. For example, by knowing which genes or proteins are correlated uniquely with CF, assessment of the clinical course for a given patient could be quantitated. Alternatively, preclinical drug discovery could be accelerated by asking whether a candidate therapeutic agent had the appropriate effects on the CF-relevant genes or proteins. In particular, probes for members of the class of CF informative genes and/or proteins could be placed on an array (e.g., a microarray or biochip) as a convenient platform for both types of analyses.

It would be greatly advantageous to the enterprise if both gene expression and protein expression correlated. However, this is not necessary for the system to function. One example in the case of CF lung epithelial cells for correlated genomics and proteomics is the proinflammatory cytokine IL-8, which has been studied in the IB-3 cell system (Eidelman et al. 2001).

The sensitivities of conventional genomic and proteomic analyses typically differ by approximately 10,000. The number can be estimated from the fact that genomic analysis easily measures ca. 10⁴ actin mRNA's, while silver staining methods need ca. 10⁸ actin protein molecules for ready detection. To detect global CF-specific gene expression (“genomics”), we have previously performed 31[P]-labeled cDNA microarray studies with RNA prepared from the CF IB3 cells and their isogenic [wildtype] CFTR-repaired IB3-S9 daughter cells (Eidelman et al. 2001). Other types of highly sensitive microarrays can also be employed (Srivastava et al. 2002). For example, in this study, fluorescence-based microarrays made by Affymetrix were also employed.

To detect global protein expression (“proteomics”), the conventional approach is to separate the proteins by 2-D gel electrophoresis and to locate the separated proteins by silver staining. As indicated above, the challenge with conventional silver stain technology is that it is not very sensitive compared with the sensitivity of genomic assays and gives only qualitative data. Other types of mass labels for proteins have been developed (e.g., fluorescent Sypro Ruby) which have the advantage of more extensive dynamic range than that of the silver stains. However, they do not provide any real advantage in terms of sensitivity or quantitation.

In the present invention, we have bypassed this problem of relative insensitivity by using 35[S]methionine to pulse-label IB-3 CF and [wildtype CFTR]-repaired IB3-S9 cells. After separating the proteins by conventional 2-D gel electrophoresis, we then image the radio-labeled proteins by autoradiography and phosphoimaging. This new data in the third dimension yields quantitative information on the individual rates of biosynthesis of each protein. We refer to this new quantitative and sensitive approach to disease proteomics as “3-D Proteomics”. We have found that nearly all silver stained features are labeled using this approach. Logically, the few methionine-free proteins that exist would have to be detected and measured with another labeled amino acid. Furthermore, we find that the present application of 3-D Proteomics delineates 3-5 fold more features than are readily located by conventional silver stain technology on a given cystic fibrosis 2-D gel.

3-D Proteomics provides a quantitative and sensitive alternative to conventional 2-D proteomics in the following ways. For example, if one knows the identify of the protein and the specific activity of the radio-label, the quantitative amount of such protein can be easily calculated. Thus 3-D Proteomics allows one to conduct a quantitative, global analysis of all biological molecules, including proteins, in a complete and quantitative manner. While we use biosynthetic labeling with 35[S] methionine in this disclosure, it is clear that other substrates could be employed. These might include other radio- or mass-labels, other amino acids; other biochemical building blocks or precursors, such as nucleotides, sugars or lipids; and inorganic species such as sulfur or phosphate to follow post-translational modifications.

In the present invention the upregulation of a cytosolic DUB, ubiquitin carboxy terminal hydrolase, type 1 (UCHL-1) in CF lung cells is described. In addition, the use of UCHL-1 as a proteomic and genomic identifier for CF lung cells is also described.

Proteomic Analysis of CF Lung Epithelial IB-3 Cells and the [Wildtype CFTR]-Repaired IB-3/S9 Cells

CF lung epithelial IB-3 cells and the [wildtype CFTR]-repaired IB-3/S9 cells were grown to ca. 80% confluence and incubated in methionine-free medium supplemented with 35[S] methionine for six hours. Cell extracts were prepared and separated by conventional 2-D gel electrophoresis. The gels were then stained with conventional silver stain, and then imaged by both autoradiography and phosphorimaging.

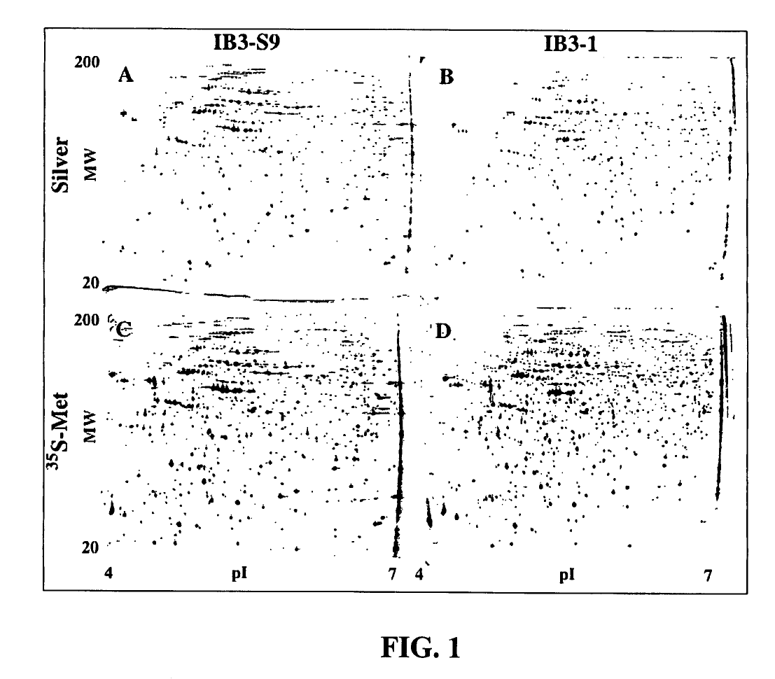

The silver stained images for both cell types are shown in FIGS. 1A and 1B, while the auto-radiographical data for both images are shown in FIGS. 1C and 1D. As can be seen from FIGS. 1A-1D, in most cases the silver-stained features are also labeled by 35[S] methionine. However, the relative radio-intensities of many of the features vary quite substantially from the staining intensities. This variation includes instances where the radiolabel occurs but the silver staining is absent.

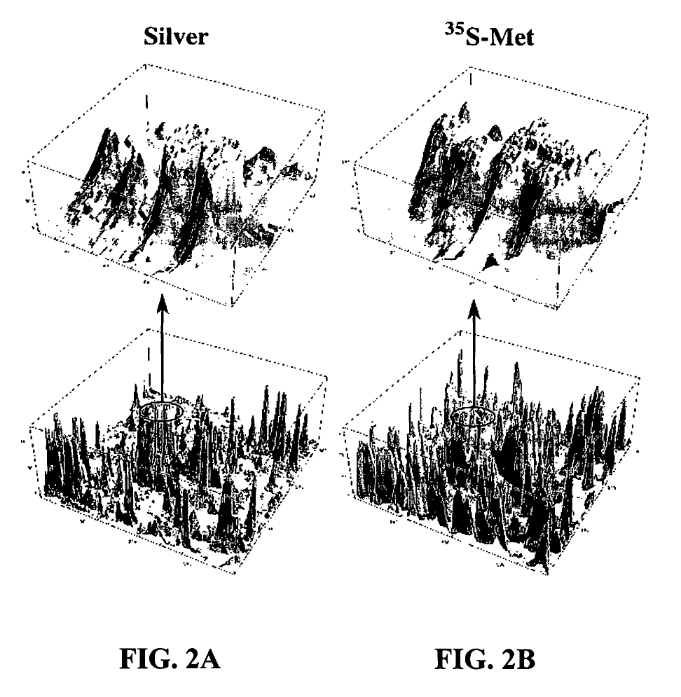

In general, the number of detectable features is 3-5 fold greater in the radio labeled images than in the same silver-stained image. The details of this difference can be readily appreciated by reference to FIGS. 2A and 2B. These figures show a comparison of the same set of features, scanned by densitometry, of both the silver-stained and 35[S] methionine labeled images of the same 2-D gel. Because the silver stain method is profoundly non-linear, low protein concentrations are hardly seen. Furthermore, because of the low dynamic range of silver stain, the higher concentrations saturate out quite quickly. This is the basis of the qualitative nature of the silver stain method. By contrast, radiolabelled images are by their nature linear over many logs and therefore show both high and low intensity features. Many of these features come to sharp points in intensity, indicating that a complete quantitative measurement has been possible. Therefore, an increased number of 35[S]-labeled features can be seen in FIG. 2B compared to the truncated silver-stained features in FIG. 2A.

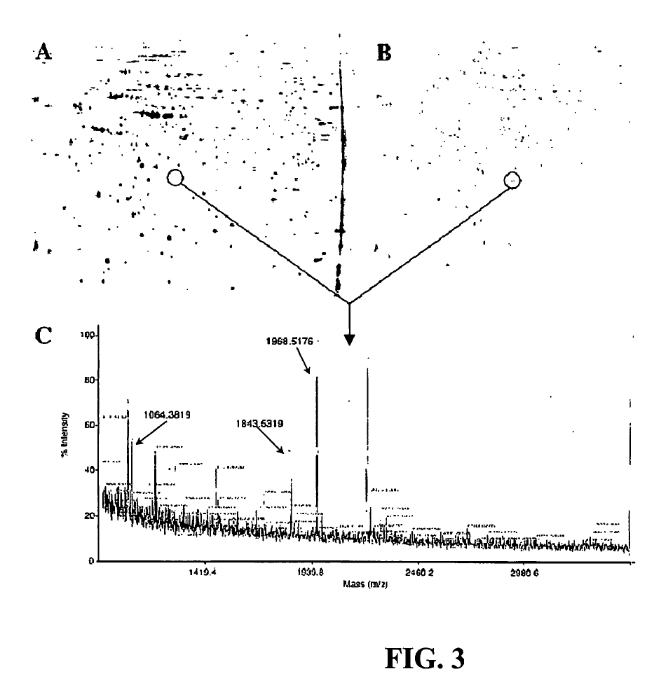

By comparing the radiolabeled images of gels for both the IB-3 and IB-3/S9 cells, we were able to conduct a systematic search for features present in one but not the other. FIGS. 3A and 3B show a side-by-side comparison of both gels differentially pseudo-color-imaged to discriminate between the samples. As only one example of such a difference, we identified the circled feature in the CF IB-3 gel in FIG. 3B, noting that the equivalent feature was absent in the same region of the gel for repaired IB-3/S9 cells (FIG. 3A). This difference can also be seen in the silver-stained gels shown in FIGS. 1A and 1B.

The circled feature was cut out and subjected to identification by mass spectrometry. The results are shown in FIG. 3C. The arrows in FIG. 3C point to prominent peptides and the corresponding numbers give their mass/charge ratios. From these data the amino acid sequences of each peptide were calculated and the identification of the circled feature as UCHL1 was made. Calculations and identifications are made by an internet-enabled database and software provided by Agilent (Hewlett-Packard, now HPQ, Framingham, Mass.). The identification is made by 100% identities between all the measured sequences and 40% of the total sequence.

As shown in FIG. 3C, the peptides corresponding to ubiquitin carboxy terminal hydrolase-1 (UCHL1; PGP9.5; ref seq # NM_{—}004181; hu chr.4p14) were detected and identified.

In the above experiment, IB-3 and IB-3/S9 cells were cultured as described by Eidelman et al. (2001). 2-D gel separations of cellular proteins were performed using the proprietary Pharmacia system which consisted of a 4-7 pH gradient in the isoelectric focusing dimension and a SDS-PAGE gel in the molecular weight dimension. Identified features were isolated and subjected to tryptic digestion using the Bio-Rad in-gel kit. Samples of the digested protein were then mixed with matrix and placed on a 10×10 planchet for analysis by mass spectrometry (MALDI-TOF, Agilent).

Genomic Analysis of CF Lung Epithelial IB-3 Cells and the [Wildtype CFTR]-Repaired IB-3/S9 Cells

Samples of RNA were prepared from IB-3 and IB-3/S9 cells and subjected to analysis on the Human Affymetrix chip. Data were analyzed by proprietary bioinformatics software to identify the most differentially expressed genes. The results of this analysis show that the UCHL1 gene was expressed at ten (10) standard deviations (SD's) greater in the CF IB-3 cell than the repaired IB-3/S9 cell. The value is based on the average difference in expression of 30,000 genes for both cell systems.

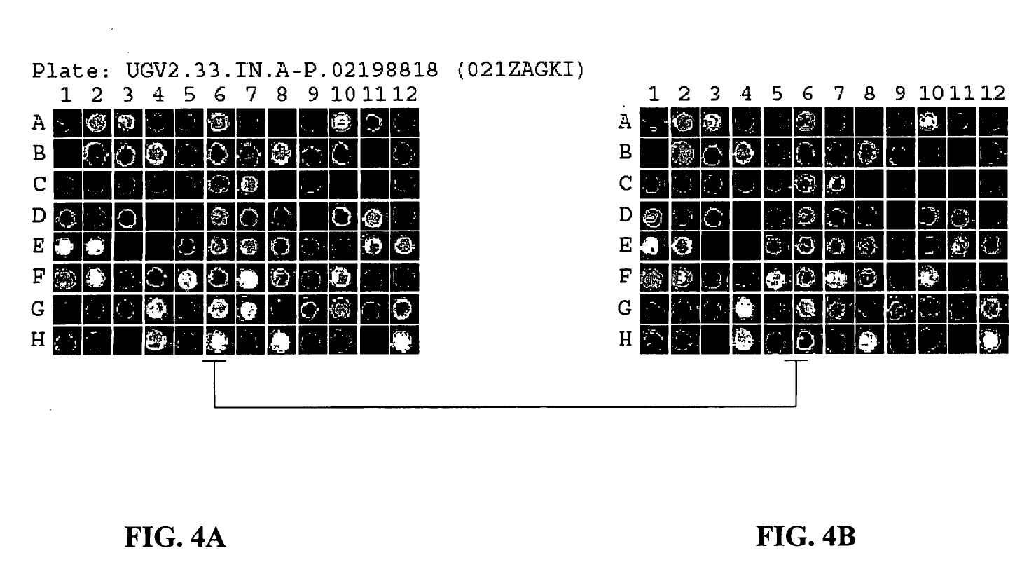

Samples of RNA from IB-3 and IB-3/S9 cells were also analyzed using an INCYTE cDNA microarray of 8000 genes. The results of this analysis showed a 3.8-fold increased level of expression of the UCHL1 gene in the IB-3 cells compared to the IB-3/S9 cells. Data for this system are shown in FIGS. 4A and 4B. The gene for UCLH-1 is located in the H-6 array position in both arrays. This array position in the IB-3 array exhibited a yellow-red psuedo-colored image while the same position in the IB-3/S9 array exhibited a blue psuedo-colored image, indicating a significantly higher level of UCLH-1 gene expression in the IB-3 cells. The raw scores for intensities are 4100 for the IB3 array and 1100 for the IB-3/S9 array.

In FIGS. 4A and 4B, RNA from the cystic fibrosis cell line IB-3 was labeled with the fluorescent compound CY3 while RNA from the wildtype CFTR-repaired cell line IB-3/S9 (labeled “S9/CY5”) was labeled with the fluorescent compound CY5. Relative expression was determined by standard methods. The pseudocolor image indicates that the IB-3 cells express more UCHL1 mRNA than the IB-3/S9 by a factor of 3.8.

In the above experiment, the RNA was prepared by standard techniques as described (Srivastava et al. 1999) and processed according to the manufacturers descriptions.

Validation of Differential Expression of UCHL1 Protein in CF Lung Epithelial IB-3 Cells and the [Wildtype CFTR]-Repaired IB-3/S9 Cells by Immunochemistry and Western Blot Analysis

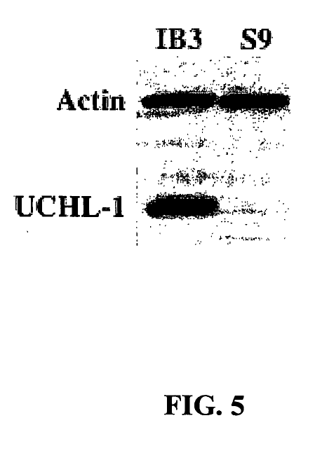

To validate the above findings, antibodies to UCHL1 were obtained and a Western blot analysis was conducted on separately prepared samples of the two cell types on 1-D SDS gels. The results are shown in FIG. 5. FIG. 5 shows a Western blot image by enhanced chemiluminescence (ECL) of UCHL-1 protein. As can be seen from FIG. 5, IB-3 cells have substantial levels of the immunodetectable UCHL1 antigen. By contrast, the repaired IB-2/S9 (denoted as “S9” in the insert) have undetectable levels of the antigen. IB-3 and IB-3/S9 cells were grown in T75 flasks, and 50 μg of protein from each cell type run on 1-D SDS-PAGE.

The gene for the ubiquitin carboxy terminal hydrolase-1 (UCHL1) is vastly overexpressed in CF lung epithelial cells at both the levels of mRNA (genomics) and protein (proteomics). The difference can be detected in terms of qualitative mass presence using silver stain on 2-D gels, or biosynthetic incorporation rate by 3-D proteomics. The observation is validated by Western blot of SDS-PAGE samples of both cell types as shown in FIG. 5.

UCHL1 is believed to stimulate proteosomic protein degradation by generating free monomeric ubiquitin. The high level of UCHL1 expression in CF cells is therefore consistent with the upregulation of proteosomic destruction of mutant [ΔF508]CFTR. Since massively elevated levels of IL-8 are found in CF lungs, this result is also consistent with enhanced proteolytic destructruction of phosphorylated IκBα. The latter process is obligatory for NFκB dependent expression of IL-8 in the CF lung.

Therefore, UCHL1 can be used as a marker for CF. For example, a probe for UCHL1 can be used to detect UCHL1 in a sample. The probe can be part of an array (e.g., a microarray). The probe can be a nucleic acid probe (e.g., cDNA or oligomer). The microarray can be used both for clinical prognosis as well as for CF drug discovery.

Although UCHL1 is specifically disclosed as a marker for CF, other genes and proteins can also be used as biomarkers for CF.

In order to determine other potential nucleic acid markers for CF, fourteen (14) patient samples were procured by bronchial biopsy and RNA from each sample prepared for analysis. IL-8 levels were measured from samples of bronchial alveolar lavage fluid from each patient. Data from these patients have been analyzed and are described below.

Table 1 summarizes data from cDNA microarray genomic analysis of ten (10) CF patients and three (3) non-CF disease control. The data shown are averages of the average data from ten CF patients for each of 1200 informative genes compared to the average of the same 1200 genes in three (3) non-CF disease control patients. The data are arranged vertically in terms of the ratio of CF genes to control genes, from largest fold increase or decrease relative to controls down to >2.0 fold. Each gene in each patient has been normalized to itself so that the actual values of gene expression in a given patient are independent of calculations for other patients. The average values for each gene are given in columns marked for CF (“CF-avg”) and for controls (“Con-avg”). The P values for each ratio are given in the column marked “sig”. Since every gene is not informative in every patient, the numbers of patients used for each analysis are given in the columns marked “Count-CF” and “Count-con”, respectively.

It should be noted that a high fold difference does not necessarily mean a significant difference. As the number of control patients increases, the P values would be expected to improve. However, there are nevertheless a number of high-fold changing genes which also have good P values. For example, the STAT-1α/β gene is 2.4 fold elevated in CF patients, P=0.029.

TABLE 1
Genes from CF and Non-CF Disease Controls Ordered According to
Decreasing Fold Difference

| Gene | Con-avg | CF-Avg | Ratio | P = sig | Count-CF | Count-Con |
|---|---|---|---|---|---|---|
| glutamate (NMDA) receptor subunit epsilon 2 precursor; N- | 1.586 | 0.973 | −4.1 | 0.084 | 10 | 3 |
| methyl D-aspartate receptor subtype 2B(NMDAR2B; | | | | | | |
| NR2B) | | | | | | |
| voltage-gated potassium channel protein KV12; HUKIV; | 0.852 | 0.371 | −3.03 | 0.136 | 10 | 3 |
| HBK5; RBK2; NGK1 | | | | | | |
| leukocyte common antigen precursor (L-CA); CD45 | 0.535 | 0.059 | −3.00 | 0.068 | 10 | 2 |
| antigen; PTPRC | | | | | | |
| CD40 receptor associated factor 1 (CRAF1) | 0.623 | 1.060 | 2.73 | 0.101 | 10 | 3 |
| adenosine A1 receptor (ADORA1) | 0.124 | 0.542 | 2.62 | 0.053 | 10 | 3 |
| SL cytokine precursor; FLT3 ligand (FLT3LG) | 0.535 | 0.937 | 2.53 | 0.130 | 10 | 3 |
| tumor necrosis factor precursor (TNF-alpha; TNFA); | 0.245 | 0.640 | 2.48 | 0.054 | 10 | 3 |
| cachectin | | | | | | |
| parkin | 0.607 | 0.215 | −2.47 | 0.137 | 10 | 3 |
| glutathione S-transferase A1 (GTH1; GSTA1); HA subunit | −0.826 | −0.448 | 2.39 | 0.119 | 6 | 2 |
| 1; GST-epsilon | | | | | | |
| signal transducer and activator of transcription 1 alpha/beta | 0.394 | 0.773 | 2.39 | 0.039 | 10 | 3 |
| (STAT1); transcription factor ISGF-3 components p91/p84 | | | | | | |
| III-1 oncogene; ergB transcription factor | 0.244 | 0.620 | 2.38 | 0.073 | 10 | 3 |
| DNA binding protein HIP116 | 0.742 | 0.367 | −2.37 | 0.258 | 10 | 3 |
| Bone Morphogenic Protein3 (BMP3) | 0.349 | −0.017 | −2.32 | 0.300 | 6 | 2 |
| translin; | 1.679 | 1.317 | −2.30 | 0.184 | 10 | 3 |
| PI3-Kinase, p110; | 0.171 | −0.177 | −2.23 | 0.240 | 6 | 2 |
| IL-2Rgamma; | 0.033 | −0.311 | −2.21 | 0.208 | 7 | 2 |
| cmyc oncogene; | 1.086 | 0.744 | −2.20 | 0.116 | 10 | 3 |
| lissencephalin X; | 0.401 | 0.064 | −2.17 | 0.086 | 10 | 3 |
| cAMP Response Element Binding Protein (CREBBP) | 0.442 | 0.108 | −2.17 | 0.054 | 9 | 3 |
| casein kinase 1 gamma 2; | 0.021 | 0.354 | 2.16 | 0.087 | 10 | 3 |
| ribosomal protein S6 kinase II alpha 3; | 0.266 | −0.064 | −2.14 | 0.160 | 9 | 2 |
| macrophage-specific colony stimulating factor (MCSF) | 0.420 | 0.084 | −2.12 | 0.163 | 10 | 3 |
| cellular retinoic acid binding protein II (CRABP2) | 1.093 | 0.768 | −2.11 | 0.087 | 10 | 3 |
| cadherin 3 (P-cadherin) | 0.225 | −0.090 | −2.06 | 0.283 | 7 | 2 |
| basic transcription factor 62-kDa subunit (BTF2) | −0.344 | −0.029 | 2.06 | 0.118 | 4 | 3 |
| placenta growth factors 1 and 2; | 0.327 | 0.019 | −2.03 | 0.125 | 10 | 3 |

Table 2 shows the same data ordered according to increasing P value. In this case, the fold change is typically lower for the listed potential targets yet the significance of the difference is higher. For example, the gene with the lowest P value is FUSE binding protein. The expression of this gene was elevated 86% in CF patients with a P value of 0.026.

TABLE 2
Genes from CF and Non-CF Disease Controls Ordered According to
P-Value (Significance)

| Gene | Con-avg | CF-Avg | Ratio | P = sig | Count-CF | Count-Con |
|---|---|---|---|---|---|---|
| FUSE binding protein | 0.561 | 0.830 | 1.86 | 0.026 | 10 | 3 |
| leukemia inhibitory factor (LIF; HILDA) | −0.286 | −0.117 | 1.48 | 0.027 | 10 | 3 |
| beta-interferon gene positive regulatory domain 1 binding | 0.510 | 0.357 | −1.42 | 0.034 | 10 | 3 |
| factor (BLIMP1); | | | | | | |
| interferon consensus sequence-binding protein (ICSBP) | −0.425 | −0.347 | 1.2 | 0.035 | 3 | 2 |
| calcium activated potassium channel HSK1 | 0.109 | −0.117 | −1.69 | 0.036 | 10 | 3 |
| c-kit proto-oncogene; mast/stem cell growth factor receptor | −0.715 | −0.535 | 1.51 | 0.039 | 6 | 2 |
| precursor (SCFR); CD117 antigen | | | | | | |
| Signal transducer and activator of transcription 1 (STAT1) | 0.394 | 0.773 | 2.39 | 0.039 | 10 | 3 |
| NFkB, p100 (NFkB, p52) | 0.068 | −0.174 | −1.76 | 0.042 | 9 | 3 |
| IL-17 | 0.041 | −0.043 | −1.21 | 0.043 | 10 | 3 |
| GABA Receptor epsilon subunit [GABA(A)Receptor] | −0.126 | 0.025 | 1.41 | 0.051 | 10 | 3 |
| Adenosine A1 Receptor (ADORA1) | 0.124 | 0.542 | 2.62 | 0.053 | 10 | 3 |
| tumor necrosis factor precursor (TNF-alpha; TNFA); | 0.245 | 0.640 | 2.78 | 0.054 | 10 | 3 |
| cachectin | | | | | | |
| cAMP-response binding protein (CREB) | 0.442 | 0.106 | −2.17 | 0.054 | 9 | 3 |
| RAB3B | −0.486 | −0.319 | 1.47 | 0.057 | 9 | 2 |
| p16-INK4 | −0.146 | 0.105 | 1.78 | 0.058 | 10 | 3 |
| frizzled | 0.115 | 0.358 | 1.75 | 0.059 | 10 | 3 |
| octamer binding transcription factor 2 (OCT-2; OTF2) | −0.435 | −0.317 | 1.31 | 0.059 | 7 | 3 |
| Interleukin 4 Precursor (IL-4) | 0.210 | −0.032 | −1.75 | 0.060 | 10 | 3 |
| Matrix metalloproteinase 12 (MMP12) | −0.218 | −0.085 | 1.36 | 0.062 | 10 | 3 |
| G-Protein activated inward rectifier Potassium channel 3 | −0.545 | −0.366 | 1.51 | 0.064 | 7 | 3 |
| (GIRK3); KIR3.3 | | | | | | |
| zinc finger protein 91 (ZNF92); HPF7; HTF10 | −0.188 | −0.091 | 1.26 | 0.064 | 10 | 3 |
| DNA Repair protein XRCC1 | −0.328 | −0.051 | 1.89 | 0.064 | 10 | 3 |
| V(D)J Recombination Activating protein RAG2 | −0.232 | −0.388 | −1.43 | 0.065 | 10 | 3 |

Based on the above analysis, any of the following genes/proteins can also be used as markers for CF according to the invention:

1. NMDA Receptor subunit epsilon 2 (NMDAR2B);

2. Voltage gated potassium channel protein KV12;

3. Leukocyte common antigen (L-CA; CD45 antigen);

4. Adenosine A1 Receptor (ADORA1);

6. Tumor Necrosis Factor alpha;

7. parkin;

8. glutathione S-Transferase A1 (GTH1);

9. Signal transducer and activator of transcription 1 (STAT1);

10. ergB;

11. DNA binding protein HIP116;

12. Bone Morphogenic Protein3 (BMP3);

13. translin;

14. PI3-Kinase, p110;

15. IL-2Rgamma;

16. cmyc oncogene;

17. lissencephalin X;

18. cAMP Response Element Binding Protein (CREBBP);

19. casein kinase 1 gamma 2;

20. ribosomal protein S6 kinase II alpha 3;

21. macrophage-specific colony stimulating factor (MCSF);

22. cellular retinoic acid binding protein II (CRABP2);

23. cadherin 3 (P-cadherin);

24. basic transcription factor 62-kDa subunit (BTF2);

25. placenta growth factors 1 and 2;

26. FUSE binding protein;

27. leukemia inhibitory factor (LIF; HILDA);

28. beta-interferon gene positive regulatory domain 1 binding factor (BLIMP1);

29. interferon consensus sequence-binding protein (ICSBP);

30. calcium activated potassium channel HSK1;

31. NFkB, p100 (NFkB, p52);

32. IL-17;

33. GABA Receptor epsilon subunit [GABA(A)Receptor];

34. RAB3B;

35. p16-INK4;

36. frizzled;

37. OCT-2;

38. IL-4;

39. Matrix metalloproteinase 12 (MMP12);

40. G-Protein activated inward rectifier Potassium channel 3 (KIR3.3);

41. zinc finger protein 91;

42. DNA Repair protein XRCC1;

43. RAG2;

44. IL-8;

45. actophilin;

46. coactosin; and

47. UCH-L1.

Any combination of the above markers can be used on an array according to the invention. The above markers can be nucleic acid markers (e.g., mRNA) or protein markers.

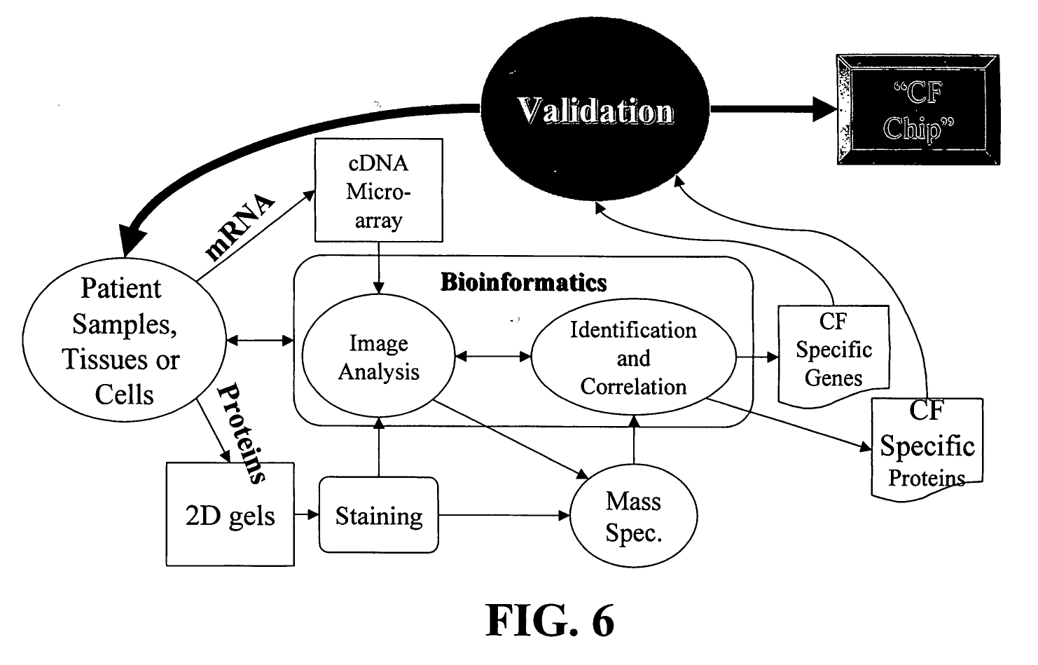

FIG. 6 is a schematic illustrating a method of identifying potential CF markers according to an embodiment of the invention. As shown in FIG. 6, proteins or mRNA from patient samples in the form of tissues or cells is analyzed using a cDNA microarray or a 2D-gel, respectively. The microarray or gel is then imaged and the resulting image analyzed using, for example, bioinformatics. In this manner, potential CF specific genes and proteins (i.e., potential markers) can be identified. Whether the potential marker is actually a CF specific marker can then be validated. A probe for the marker can then be used in an array (e.g., a microarray).

The probes can be any polynucleotide (i.e., a nucleic acid) or polypeptide capable of binding to a target nucleic acid or protein. For example, when nucleic acid markers are used, the probes can be oligonucleotide or cDNA probes. Alternatively, when protein markers are used, the probes can be polypeptides (e.g., antibodies) or nucleic acids (e.g., aptamers). The probes can also be peptide nucleic acids in which the constituent bases are joined by peptide bonds rather than phosphodiester linkages.

The array can comprise probes for both nucleic acid and protein CF targets. For example, the array can have nucleic acid probes (e.g., cDNA or oligonucleotide probes) for CF mRNA targets as well as aptamer probes for CF protein targets attached to the same solid support. The array can comprise probes for a particular CF mRNA as well as its corresponding protein.

More than one probe can be used for each target molecule. For example, when the target molecule is mRNA, two or more cDNA or oligonucleotide probes can be used each of which is capable of hybridizing to a different subsequence of the mRNA target.

The nucleic acid probes (e.g., cDNA or oligonucleotide probes) can be complementary or substantially complementary to a subsequence of the target nucleic acid. Substantially complementary refers to the presence of minor mismatches that can be accommodated by reducing the stringency of the hybridization media. Generally, the probes are capable of hybridizing to the target molecule under the hybridization conditions employed. The length of the probes can be varied to achieve the desired level of hybridization and specificity of binding.

The array can be used in assays for diagnosing cystic fibrosis or determining the prognosis of a pateint with CF. Thus, according to one embodiment of the invention, a method is provided which includes: removing cells from a patient; lysing the cells; and contacting the cell lysate with an array as set forth above. The cells can be taken from a sputum sample. Alternatively, the cells can be white blood cells or epithelial lung cells. The epithelial lung cells can be harvested from the lung of a patient using a small brush. The method as set forth above can further include imaging the array.

The patient can be a patient that has not been diagnosed with cystic fibrosis wherein the method is a method for diagnosing cystic fibrosis. The method can further include comparing the image of the array as set forth above with a control image made by imaging an array contacted with a control composition comprising the lysate of a cell having the wildtype CFTR gene. Alternatively, the patient can be a patient that has been diagnosed with cystic fibrosis wherein the method is a method for determining the prognosis of the disease. According to a further embodiment, the patient can be a patient undergoing treatment for cystic fibrosis wherein the method is a method for determining the effectiveness of the treatment. The method can further include comparing the image of the array as set forth above with a control image made by imaging an array contacted with a control composition comprising the lysate of a cell sample removed from the patient during an earlier stage of the treatment.

The array can be used in assays for drug screening. Thus, according to a further embodiment of the invention, a method is provided which includes: contacting cells having a mutated form of the CFTR gene with a composition comprising a test compound; lysing the cells; and contacting the cell lysate with an array as set forth above. This method can further include imaging the array and comparing the image of the array with a control image made by imaging an array contacted with a control composition which does not include the test compound.

While the foregoing specification teaches the principles of the present invention, with examples provided for the purpose of illustration, it will be appreciated by one skilled in the art from reading this disclosure that various changes in form and detail can be made without departing from the true scope of the invention.

### REFERENCES

Bonfield, T. L., Konstan, M. W., Burfeind, P., Panuska, J. R., Hilliard, J. B., and Berger, M. (1995a) Normal bronchial epithelial cells constitutively produce the anti-inflammatory cytokine interleukin 10, which is downregulated in cystic fibrosis. Am. J. Respir. Mol. Biol. 13:257-261.
Bonfield, T. L., Panuska, J. R., Konstan, M. W., Hilliard, K. A., Hilliard, J. B., Ghnaim, H., and Berger, M. (1995b) Inflammatory cytokines in cystic fibrosis lungs. Am. J. Respir. Crit. Care Med. 152:2111-2118.
Briars, G. L., Dean, T. P., Murphy, J. L., Rolles, C. J., and Warner, J. O. (1995) Faecal interleukin-8 and tumour necrosis factor-alpha concentrations in cystic fibrosis. Arch. Dis. Child 73:74-76.
D'Acquisto, F., May, M. J., and Ghosh, S. (2002) The ever-widening spectrum of NF-kB inhibitors. Molec. Interv. 2:22-35.
Eidelman, O., Srivastava, M., Zhang, J., Murthy, J., Heldman, E., Jacobson, K. A., Metcalfe, E., Weinstein, D., and Pollard, H. B. (2001a) Genes from the TNFaR/NFkB pathway control the pro-inflammatory state in cystic fibrosis epithelial cells. Molecular Medicine 7:523-534
Hatakeyama, S., Kitagawa, M., Nakayama, K., Shirane, M., Matsumoto, M., Hattoriu, K., Higashi, H., Nakano, H., Okumura, K., Onoe, K., and Good,
R. A. (1999) Ubiquitin-dependent degradation of IkappaBalpha is mediated by a ubiquitin ligase Skp1/Cul1/F-box protein FWD1 Proc. Nat. Acad. Sci. (USA) 96:859-863.
Hershko, A., and Ciechanover, A. (1998) The ubiquitin system. Annu. Rev Biochem. 67:425-479.
Kitagawa, M., Hatakeyama, M., Shirane, M., Matsumoto, M., Ishida, N., Hattori, K., Nakamichi, I., Kikuchi, A., and Nakayama, K. (1999) An F-box protein, FWD1, mediates ubiquitin-dependent proteolysis of beta-catenin. EMBO J. 18:2401-2410.
Kiyosawa, H., Harada, T., Ichihira, N., Wakana, S., Kikuichi, T., and Wada, K. (1999) Intragenic deletions in the gene encoding ubiquitin carboxy terminal hydrolase in gad mice. Nature Genetics 23: 10-11.
Larsen, C. N., Krantz, B. A., and Wilkinson, K. D. (1998) Substrate specificity of deubiquitnylating enzymes: ubiquitin carboxy terminal hydrolase. Biochemistry 37:3358-3368.
Plemper, R. K. and Hammond, A. L. (2002) Protein degradation in human disease. In Protein Degradation in Health and Disease. (Progress in Molecular and Subcellular Biology, ed. M. Reboud-Ravaux) 29:61-84. Springer, Berlin and elsewhere.
Srivastava, M., Eidelman, O., and Pollard, H. B. (1999) Pharmacogenomics of the Cystic Fibrosis Transmembrane Conductance Regulator (CFTR) and the Cystic Fibrosis Drug CPX using genome microarray analysis. Molecular Med. 5:753-767.
Srivastava, M., Eidelman, O., and Pollard, H. B. cDNA Microarray for pharmacogenomic Analysis of Cystic Fibrosis. (Editor, W. Skach) Methods in Molecular Medicine 70: 21-29, 2002.
Voges, D., Zwickl, P., and Baumeister, W. (1999) The 26S proteosome: a molecular machine designed for controlled proteolysis. Annu. Rev Biochem 68:1015-1068.
Yaron, A., Hatzubai, A., Davis, M., Lavon, I., Amit, S., Manninmg, A. M., Anderson, J. S., Mann, M., Mercurio, F. and Ben-Neriah, Y. (1998) Identification of the receptor component of the IkBa-ubiquitin ligase Nature 396:590-594.
Winston, J. T., Strack, P., Beer-Romero, P., Chu, C. Y., Elledge, S. J., and Harper, J. W. (1999) The SCFb-TRCP-ubiquitin ligase complex associates specifically with phosphorylated destruction motifs in IkBa and b-catinin and stimulates IkBa ubiquitinylation in vitro. Genes and Devel. 13:270-283.

What is claimed is:

1. A method of determining the level of expression of a population of proteins in a first cell comprising:
growing the first cells in a medium comprising radio-labeled amino acids such that the radio-labeled amino acid is incorporated into the proteins;
lysing the first cells;
placing the first cell lysate on a first gel;
separating the proteins in the first cell lysate using 2-D gel electrophoresis; and
imaging the first gel using autoradiography.

2. The method of claim 1, wherein the first cells have a mutated form of the CFTR gene.

3. The method of claim 1, wherein the cells have the wildtype CFTR gene.

4. The method of claim 2, further comprising:
growing second cells having the wildtype CFTR gene in methionine-free medium supplemented with 35[S] methionine;
lysing the second cells;
placing the second cell lysate on a second gel;
separating the proteins in the second cell lysate using 2-D gel electrophoresis;
imaging the second gel using autoradiography; and
comparing the images for the first and second cells.

5. The method of claim 4, wherein the first cells are IB-3 cells and wherein the second cells are IB-3/S9 cells.

6. The method of claim 4, further comprising:
identifying proteins that exhibit different levels of expression between the first and second cells.

7. The method of claim 6, further comprising:
identifying the nucleotide sequence of aptamers which bind the identified proteins.

8. The method of claim 7, further comprising:
constructing an array comprising aptamers having the identified sequences attached to a solid support.

9. The method of claim 1, wherein the radio-labeled amino acid is 35[S] methionine.

10. The method of claim 1, wherein the population of proteins is the proteome of the first cell.

11. An array comprising:
a plurality of different probes disposed on a surface of a solid support, wherein each of the different probes bind to a different marker for cystic fibrosis.

12. The array of claim 11, wherein the plurality of different probes include probes for UCHL-1 and IL-8.

13. The array of claim 11, wherein the probes and markers are nucleic acids.

14. The array of claim 11, wherein the probes comprise cDNA or oligonucleotide probes.

15. The array of claim 11, wherein the markers comprise mRNA markers.

16. The array of claim 11, wherein the probes comprise nucleic acid probes and the markers comprise protein markers.

17. The array of claim 15, wherein the probes comprise aptamers.

18. The array of claim 1, wherein the markers are selected from the group consisting of: NMDA Receptor subunit epsilon 2 (NMDAR2B); Voltage gated potassium channel protein KV12; Leukocyte common antigen (L-CA; CD45 antigen); Adenosine A1 Receptor (ADORA1); CD40 Receptor Associated Antigen (CRAF-1); Tumor Necrosis Factor alpha; parkin; glutathione S-Transferase A1 (GTH1); Signal transducer and activator of transcription 1 (STAT1); ergB; DNA binding protein HIP116; Bone Morphogenic Protein3 (BMP3); translin; PI3-Kinase, p110; IL-2Rgamma; cmyc oncogene; lissencephalin X; cAMP Response Element Binding Protein (CREBBP); casein kinase 1 gamma 2; ribosomal protein S6 kinase II alpha 3; macrophage-specific colony stimulating factor (MCSF); cellular retinoic acid binding protein II (CRABP2); cadherin 3 (P-cadherin); basic transcription factor 62-kDa subunit (BTF2); placenta growth factor 1; placenta growth factor 2; FUSE binding protein; leukemia inhibitory factor (LIF; HILDA); beta-interferon gene positive regulatory domain 1 binding factor (BLIMP1); interferon consensus sequence-binding protein (ICSBP); calcium activated potassium channel HSK1; NFkB, p100 (NFkB, p52); IL-17; GABA Receptor epsilon subunit [GABA(A)Receptor]; RAB3B; p16-INK4; frizzled; OCT-2; IL-4; Matrix metalloproteinase 12 (MMP12); G-Protein activated inward rectifier Potassium channel 3 (KIR3.3); zinc finger protein 91; DNA Repair protein XRCC1; RAG2; IL-8; actophilin; coactosin; UCH-L1 and combinations thereof.

19. A method comprising:
removing cells from a patient;
lysing the cells; and
contacting the cell lysate with an array as set forth in claim 1.

20. The method of claim 19, further comprising imaging the array.

21. The method of claim 19, wherein the patient has not been diagnosed with cystic fibrosis and wherein the method is a method for diagnosing cystic fibrosis.

22. The method of claim 20, wherein the patient has not been diagnosed with cystic fibrosis and wherein the method is a method for diagnosing cystic fibrosis.

23. The method of claim 22, further comprising comparing the image of the array with a control image made by imaging an array contacted with a control composition comprising the lysate of a cell having the wildtype CFTR gene.

24. The method of claim 19, wherein the patient has been diagnosed with cystic fibrosis and wherein the method is a method for determining the prognosis of the disease.

25. The method of claim 19, wherein the patient is undergoing treatment for cystic fibrosis and wherein the method is a method for determining the effectiveness of the treatment.

26. The method of claim 20, wherein the patient is undergoing treatment for cystic fibrosis and wherein the method is a method for determining the effectiveness of the treatment.

27. The method of claim 26, further comprising comparing the image of the array with a control image made by imaging an array contacted with a control composition comprising the lysate of a cell sample removed from the patient during an earlier stage of the treatment.

28. A method comprising:
contacting cells having a mutated form of the CFTR gene with a composition comprising a test compound;
lysing the cells;
contacting the cell lysate with an array as set forth in claim 1.

29. The method of claim 28, further comprising imaging the array.

30. The method of claim 28, further comprising comparing the image of the array with a control image made by imaging an array contacted with a control composition which does not include the test compound.
